(19) United States
(12) Patent Application Publication
(10) Pub. No.: US 2021/0244276 A1
(43) Pub. Date: Aug. 12, 2021

(54) SPECTRALLY ADJUSTABLE OPTICAL PHOTOSENSITIVITY ANALYZER AND USES THEREOF

(71) Applicant: Johnson & Johnson Vision Care, Inc., Jacksonville, FL (US)

(72) Inventors: Derek Nankivil, Jacksonville, FL (US); Jean-Marie A. Parel, Miami, FL (US)

(21) Appl. No.: 17/112,245

(22) Filed: Dec. 4, 2020

Related U.S. Application Data
(60) Provisional application No. 62/944,991, filed on Dec. 6, 2019.

(51) Int. Cl. A61B 3/14; A61B 5/00

(52) U.S. Cl. CPC A61B 3/145; A61B 3/0008; A61B 5/0077

(57) ABSTRACT

A spectrally adjustable ocular photosensitivity analyzer (SAOPA) is capable of emulating light sources common in everyday environments. An array of multiple light sources generates the desired spectra at intensities that are sufficient to elicit an uncomfortable level of photostress or light discomfort in normal human subjects sufficient to identify, and preferably quantify, a visual photosensitivity threshold of a human subject.

### I. CROSS REFERENCE TO RELATED APPLICATIONS

This application claims priority to U.S. Prov. App. No. 62/944,991, filed Dec. 6, 2019, the entire disclosure of which is incorporated by reference herein for all purposes. All publications and patent applications mentioned in this specification are herein incorporated by reference to the same extent as if each individual publication or patent application was specifically and individually indicated to be incorporated by reference.

### II. BACKGROUND OF THE INVENTION

### a. Field of the Invention

Embodiments relate generally to systems for analyzing ocular photosensitivity in human subjects and methods of using such devices. More particularly, embodiments described herein relate to a spectrally adjustable optical photosensitivity analyzer (SAOPA) capable of emulating light sources common in everyday environments (also referred to as ecological light sources), including solar, halogen, fluorescent, xenon, incandescent or other common light sources. Use of a SAOPA such as those claimed and described herein with human subjects may permit the detailed characterization of the role of spectra and color on ocular photostress.

### b. Background and Discussion of the Related Art

It is approaching one hundred years since Holladay (Holladay, 1926) and Stiles (Stiles, 1929) produced seminal manuscripts indicating that bright illumination conditions can produce two glare effects: glare discomfort and glare disability. Glare discomfort generally refers to the condition where discomfort or even pain is experienced when exposed to bright light. Glare disability refers to a reduction in visibility of visual function due to the presence of bright light. Photosensitivity is a sensitivity or pain in response to light and is related to a number of ocular disorders including: dry eye, blepharospasm, migraine, traumatic brain injury, achromatopsia, retinitis pigmentosa, macular pigment epithelium atrophy, retinal ganglion cell hypertrophy or degeneration, iris muscle atrophy, IOL dysphotopsia, and others. Although the term photosensitivity often refers to an ocular disorder, given the association with light induced pain or discomfort, the terms photosensitivity and glare discomfort are sometimes used interchangeably.

Photostress is generally understood as the aftermath of extreme glare disability. After being exposed to intense illumination, it takes time for one's visual system to readjust sensitivity to the new conditions. A filter that reduces the magnitude of the photostress will expedite photostress recovery. Recently, photosensitivity thresholds (PTs) were measured using an ocular photosensitivity analyzer (OPA) consisting of a bank of computer-controlled light emitting diodes (LEDs). The stimulus intensity was varied in a staircase procedure adapted for each subject's response to determine the PT. However, this system is limited by the fixed optical properties of the white LEDs and thus incapable of emulating the various differing spectral characteristics associated with ecologically valid stimuli. Accordingly, there remains a need for improved systems for analyzing ocular photosensitivity capable of, among other things, more accurately emulating lighting conditions encountered in daily life and identifying, and preferably quantifying, a visual photosensitivity threshold of a human subject.

### III. SUMMARY OF THE INVENTION

Disclosed herein are ophthalmic systems and methods capable of presenting retinal stimuli with ecologically valid spectra and a psychophysical paradigm for assessing photosensitivity or discomfort thresholds.

In an embodiment, an ocular photosensitivity analysis system includes a light panel configured to cast light toward an eye of a human subject comprising an array of light sources having different wavelengths selected such that light emitted from the array of light sources combine to emulate a light emission spectra of an ecological light source; and an imaging system comprising a camera configured to capture images of at least a portion of an eye of a human subject in response to exposure to the light emitted from the array of light sources.

1) In a further embodiment, a method includes quantifying a visual photosensitivity threshold of a human subject employing a spectrally adjustable ocular photosensitivity analysis system as, the method including emitting light toward an eye of the human subject at increasing intensities beginning with a least light intensity and increasing toward a greatest light intensity; receiving a stimulus response from the human subject indicating at what intensity the light causes discomfort; and repeating the foregoing to achieve a plurality of reversals, i.e., a change of the subject's current response is different from the previous stimulus response, changing from yes (positive) to no (negative) or vice versa.

### IV. BRIEF DESCRIPTION OF THE DRAWINGS

2) The foregoing and other features and advantages of the invention will be apparent from the following, more particular description of preferred implementations of the invention, as illustrated in the accompanying drawings.

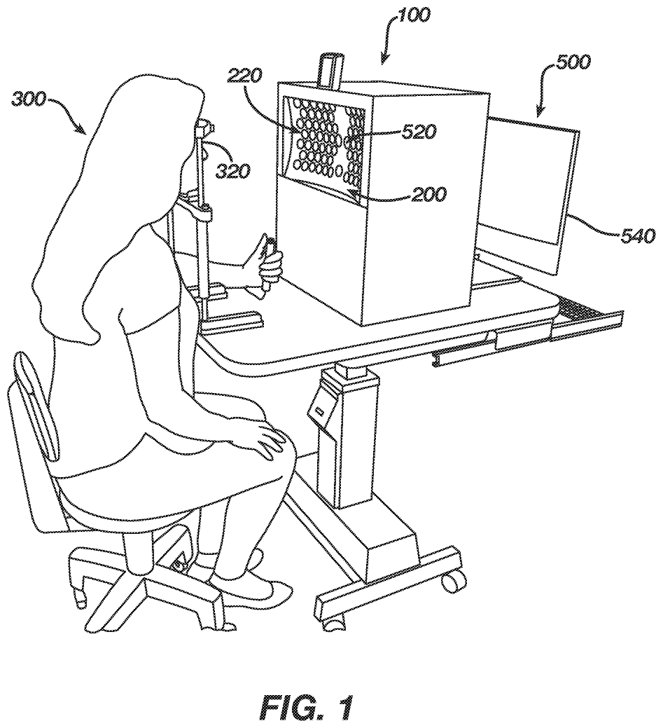

3) FIG. 1. illustrates a perspective view of a spectrally adjustable optical photosensitivity analyzer (SAOPA) in accordance with an embodiment.

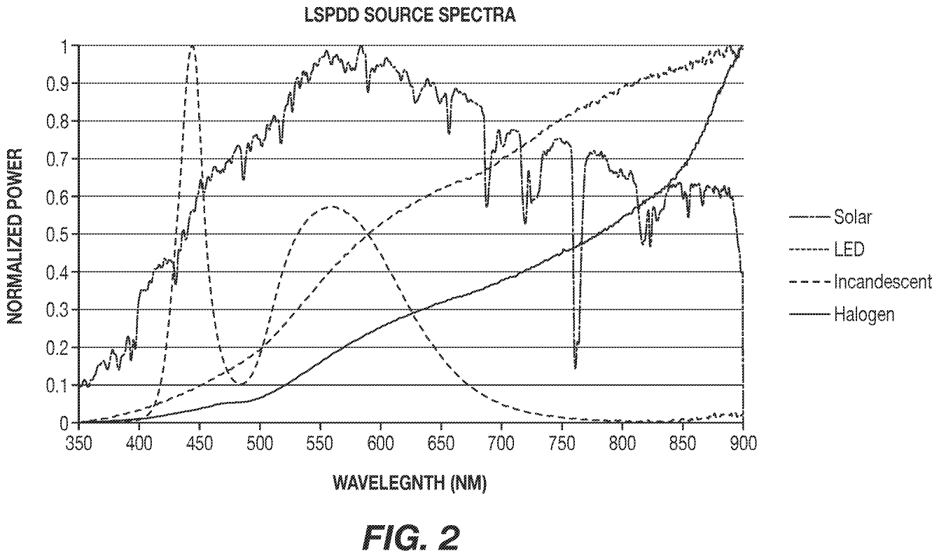

4) FIG. 2. presents a spectral power distribution of light emissions from a variety of ecological light sources.

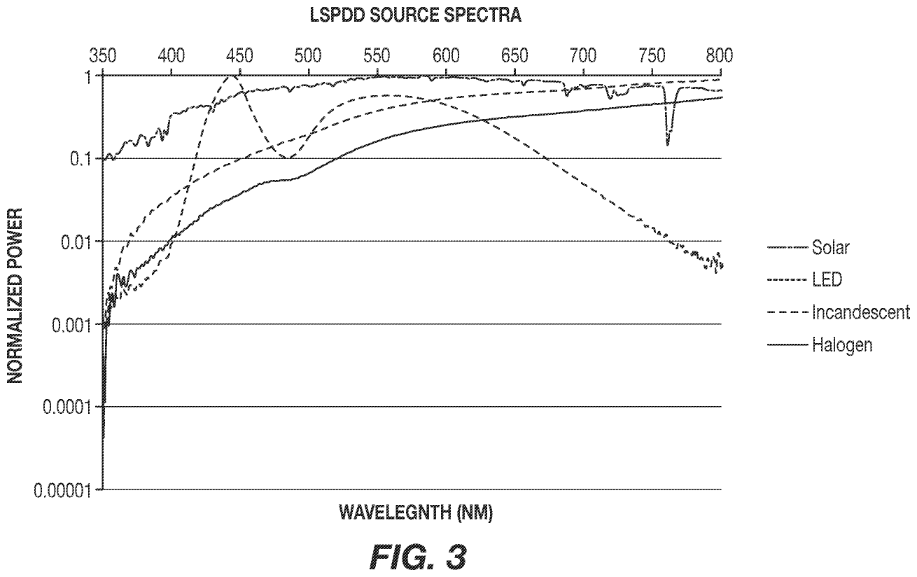

5) FIG. 3. presents a spectral power distribution of light emissions from a variety of ecological light sources in logarithmic scale.

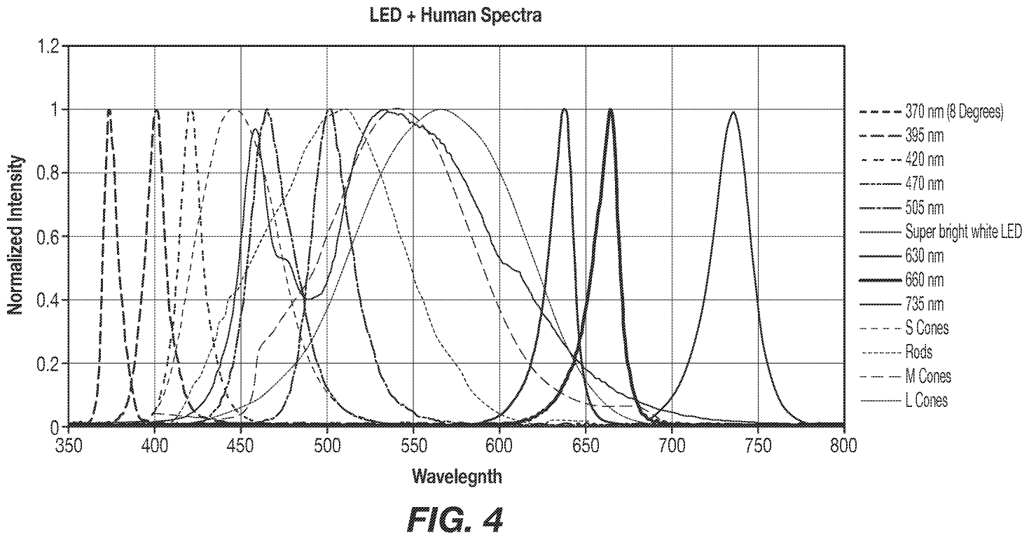

6) FIG. 4. presents an exemplary selection of light emitting diodes' spectra and intensity according to an embodiment.

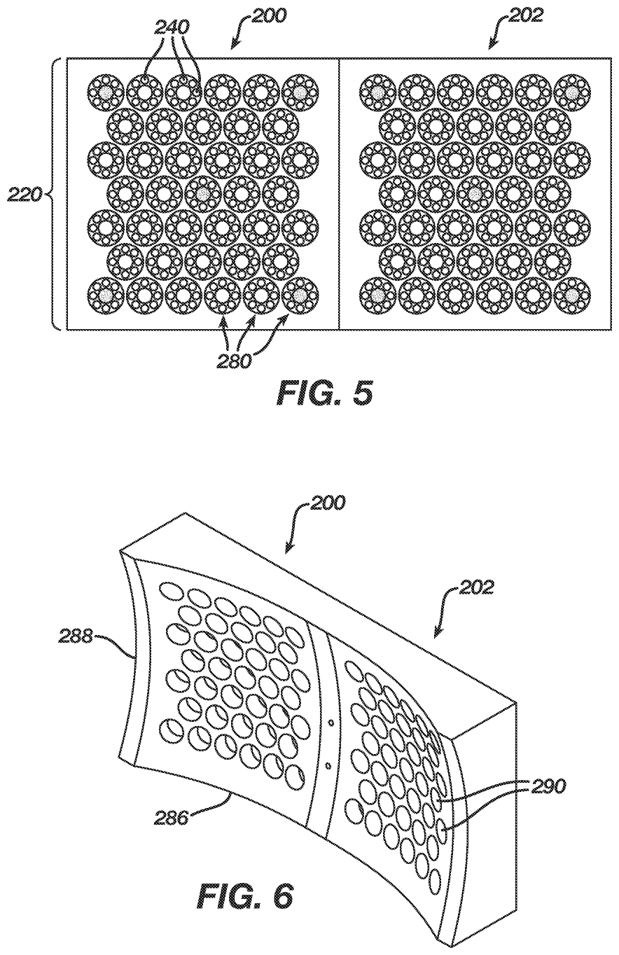

7) FIG. 5. illustrates a light panel and embedded light sources in accordance with an embodiment.

8) FIG. 6. illustrates a light panel having a bicupola shape in accordance with an embodiment.

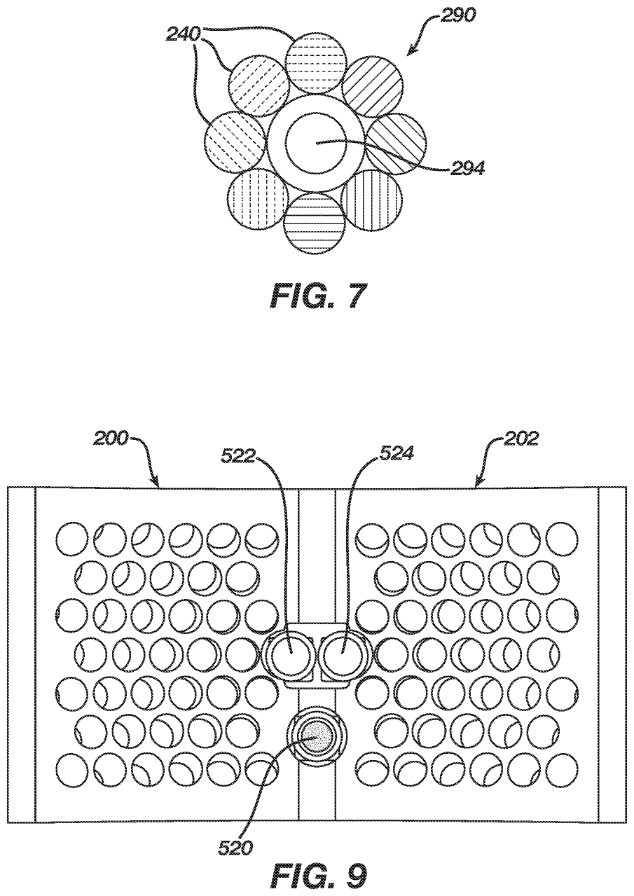

9) FIG. 7. illustrates a subarray of light sources arranged in a mini-flower configuration in accordance with an embodiment.

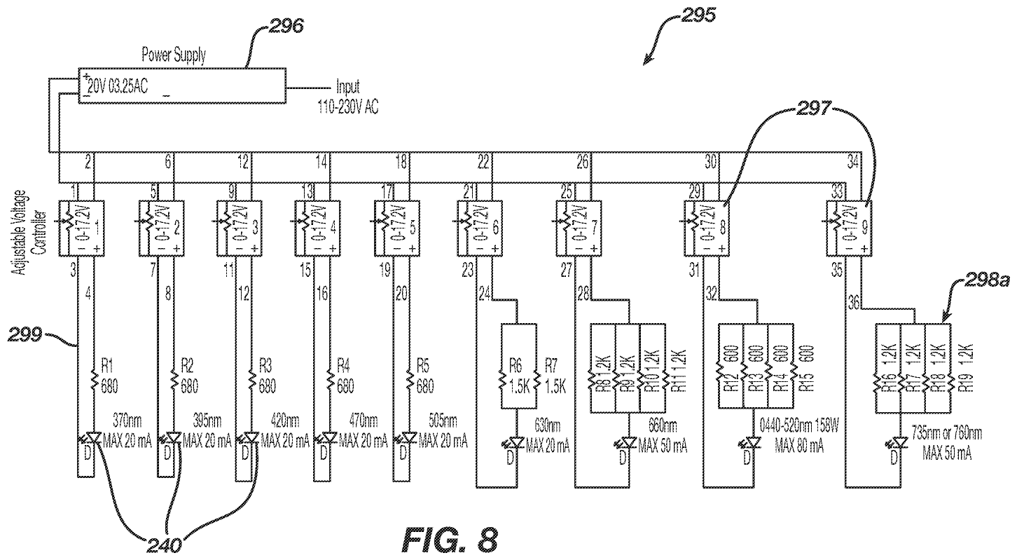

10) FIG. 8. illustrates an electrical schematic for a sub array of light sources in accordance with an embodiment.

11) FIG. 9. illustrates a light panel incorporating a camera system in accordance with an embodiment.

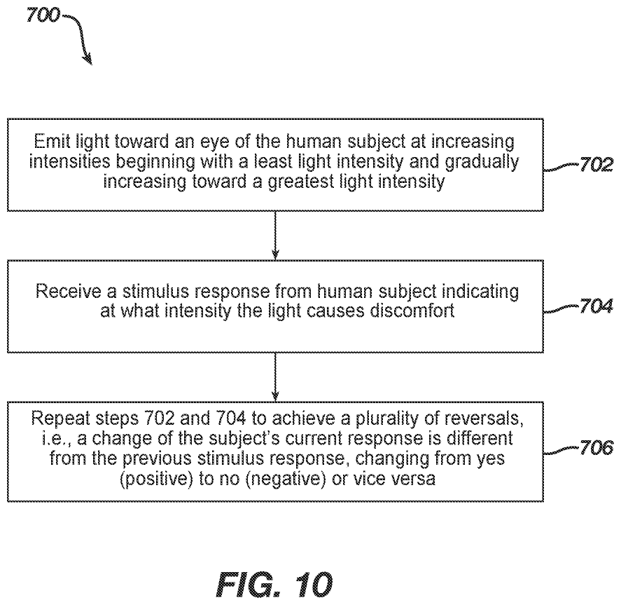

12) FIG. 10 illustrates a process for quantifying a visual photosensitivity threshold of a human subject using a SAOPA.

### V. DESCRIPTION

The foregoing and other features and advantages of the invention will be apparent from the following, more particular description of preferred embodiments of the invention, as described herein.

13) FIG. 1. illustrates a perspective view of a spectrally adjustable optical photosensitivity analyzer (SAOPA) 100 in accordance with an exemplary embodiment. The SAOPA 100 is a device capable of identifying, and preferably quantifying, a visual photosensitivity threshold of a human subject 300. The SAOPA 100 includes a light panel 200 that houses an array of light sources 220. The light panel 200 is configured to cast light toward a human subject 300 and, in particular, the eyes 320 of the subject. The light sources in the array of light sources 220 are selected to emit wavelengths such that light emitted combine to emulate a light emission spectra of an ecological light source. When combined, it is desirable, although not required, that the array of light sources 220 produce 32,000 lux at full power in order to simulate a wide range of ecologically valid stimuli. As used herein, an ecological light source means a light source encountered in human environment and includes but is not limited to solar, halogen, fluorescent, xenon, and incandescent light. As described in more detail herein, the SAOPA 100 is configured to emulate ambient lighting conditions using light sources, preferably light emitting diodes (LEDs), although other light sources including (filtered superluminescent, incandescent, supercontinuum, etc.) are possible as will be appreciated by one of skill in the art, in order to quantify the effect of high-pass spectral filters with varying cutoffs on optical photosensitivity under ecologically valid illumination. SAOPA 100 also includes an imaging system 500 that includes a camera 520 configured to capture images of at least a portion of an eye 320 of the subject 300 in response to exposure to the light emitted from the array of light sources 220. Imaging system 500 also utilizes a computing system 540 in communication the camera 520 and operative capture to still images and preferably high resolution video and/or high resolution infrared video to record light intensity data, track the interval between stimuli, record subject responses, and calculate the light intensity and LED voltage coefficients. Computing system 540 is further operable to execute a testing protocol capable of quantifying a visual photosensitivity threshold of the human subject, as described in more detail herein.

14) It is desirable to configure the light panel 200 of the SAOPA 100 such that the human subject 300 is positioned such that the intensity of the light reaching the retina of the eye 320 has sufficient luminance to support the protocols described further herein with reference to FIG. 10, yet remain within the field of view of the subject 300. The illuminance of light emitted from the array of light sources decreases by the inverse square law (intensity ∝1/distance²) where distance is defined as the distance between the light panel and the eye 320 of the human subject. As the LED panel light source is placed closer to the subject 300, light sources at the more peripheral region of the light panel 200 may only reach the far retina periphery of the eye 320 or may fall outside of the field of view. One gauge of whether a distance between the eye 320 and the light panel 200 is the number of after images experienced by a human subject, where “after images” are defined as the quantity of white spots observed by a subject after being exposed to light from the source array for a given period (e.g., one minute). As light begins to fall outside of the subject's field of view, fewer after images will be produced than when the panel is placed further away from the subject. In the example embodiment disclosed herein, a distance of 350 mm between the center of the light panel and the eye was selected, however, other distances are possible, including but not limited distances within a preferred range of between about 350 mm and 500 mm.

15) Turning to FIG. 2, a representative spectral power distribution of light emissions is provided for a variety of ecological light sources suitable for an embodiment of the invention. As noted, the SAOPA desirably has the ability to emulate one or preferably more ecologically valid light sources. The chart plots normalized power relative to a continuum of wavelengths along the visible spectrum for a variety of exemplary ecological lights sources, namely solar, LED, incandescent, and halogen lighting. Relevant spectral data may be sourced, for example, from the publicly available Light Spectral Power Distribution Database (LSPDD) by Johanne Roby, Ph.D. and Martin Aubé, Ph.D. The LSPDD is a spectral database that includes several types of artificial lighting such as public, domestic, and light therapy sources. FIG. 3. presents the same spectral power distribution data from a variety of ecological light sources in logarithmic scale.

16) As noted previously, it is desirable that light emitted from the array of light sources of the SAOPA combine to emulate the light emission spectra of a variety of ecological light sources. An embodiment capable of emulating each of solar, LED, incandescent, and halogen, may be achieved using combinations of multiple LEDs selected as shown in FIG. 4. In such an embodiment, the wavelengths of the selected light sources may include light sources with wavelengths falling the following ranges: about 370 nm, about 395 nm, about 420 nm, about 470 nm, about 505 nm, about 545 nm, about 630nm, about 660 nm, and about 735 nm. Furthermore, spectral characteristics of each of the light sources may be selected to permit metameric representation across a wide color gamut, where two stimuli are metameric when they are perceived as the same color despite having different spectral power distributions.

17) FIG. 5. illustrates light panel 200 and embedded light sources 220 in accordance with an embodiment. In this example, an array of light sources 220 are embedded into the light panel in multiple sub arrays 280 each composed of multiple LEDs 240. As described further in reference to FIG. 6 below, light panel 100 may be configured in a cupola shape as in the exemplary embodiment disclosed herein. Further, SAPOA 100 may include a second light panel 202 that substantially mirrors the configuration of light panel 200. The plurality of subarrays of light sources 280 in this example embodiment was selected at 78 subarrays (each configured in a mini-flower arrangement as described in more detailed below with reference to FIG. 7.). It should be noted, however, that subarrays may be arranged in any number of configurations including other mosaic patterns wherein each of the light sources in each of the sub arrays emits a light of a different wavelength. One such beneficial arrangement includes a hexagonal configuration in which a central primary light source is surrounded by peripheral light sources in a generally hexagonal arrangement to optimize fill factor in the array. With identical sized circular array elements, the densest possible packing (greatest fill factor) is achieved when the array elements are arranged in a hexagonal packing arrangement.

18) FIG. 6. further illustrates light panels 200 and 202 configured in a mirroring bicupola shape. In this embodiment, the light panel and the second light panel each have a horizontal radius 286 and vertical radius 288 that point to an average interpupillary distance of preferably about 32 mm from the center of the face of the human subject when the light center of the light panel is position 350 mm from the subject. Light panels 200 and 202 include openings 290 into which light sources or arrays thereof may be embedded in the panel. In the exemplary embodiment herein, light panels 200 and 202 each contain 39 openings positioned and sized to house 79 subarrays as described above. Light panels 200 and 202 may be fabricated as a single or separate components and may be cast molded, 3D printed, or other means recognizable to one skilled in the art. Suitable materials include, polylactic acid (PLA), ASA, ABS, PLA, Nylon, polycarbonate, and other plastics or metals capable of maintaining material integrity.

19) Turning now to FIG. 7. is an illustration of a subarray of light sources arranged in a mosaic pattern that forms a mini-flower configuration 290 in accordance with the exemplary embodiment disclosed herein. As used herein, a mini-flower configuration refers to a mosaic pattern where a central primary light source is surrounded by an annulus of multiple peripheral light sources. In the example embodiment shown, the mini-flower configuration is composed of a central bright white LED and eight peripheral LEDs each emitting a different wavelength of light. In the particular example embodiment set forth herein, a listing of selected specific LEDs is provided in the below table:

TABLE 1
20)

| 21) | 22) Part | 23) Viewing | 24) Power | 25) LED | |
|---|---|---|---|---|---|
| Wavelength | Number | Half Angle | Output | size | [symbol] |
| 27) 370 nm | 28) XSL0370 | 29) 7.5 | 30) 4-6 | 31) 5 mm | |
| | SE | | mW @ | | |
| | | | 20 mA | | |
| 33) 395 nm | 34) LED395- | 35) 8 | 36) 11 | 37) 5 mm | [symbol] |
| | 01V | | m@@ | | |
| | | | 20 mA | | |
| 39) 420 nm | 41) LED420- | 42) 8 | 43) 15 mW | 44) 5 mm | [symbol] |
| 40) | 01 | | @ | | |
| | | | 20 mA | | |
| 46) 440/520 | 47) YSL- | 48) 15 | 49) 16-20 | 50) 10 mm | [symbol] |
| nm peaks | R1042WC | | cd @ | | |
| | | | 20 mA | | |
| 52) 470 nm | 53) B4B-437- | 54) 4 | 55) 3.8 cd | 56) 5 mm | [symbol] |
| | IX | | @ | | |
| | | | 20 mA | | |
| 58) 505 nm | 59) B5-433- | 60) 7.5 | 61) 8.5 cd | 62) 5 mm | [symbol] |
| | B505 | | @ | | |
| | | | 20 mA | | |
| 64) 630 nm | 65) B5B-435- | 66) 4 | 67) 13.5 cd | 68) 5 mm | [symbol] |
| | TL | | @ | | |
| | | | 20 mA | | |
| 70) 660 nm | 71) LED660N- | 72) 12 | 73) 15 mW | 74) 5 mm | [symbol] |
| | 03 | | @ | | |
| | | | 50 mA | | |
| 76) 735 nm | 77) LED735- | 78) 10 | 79) 18 mW | 80) 5 mm | [symbol] |
| | 01AU | | @ | | |
| | | | 50 mA | | |

[symbol]

indicates data missing or illegible when filed

82)

[symbol]

83) In this case, the central super-bright-white LED is represented by the Sparkfun YSL-R1042WC 10 mm LED whereas the eight remaining 5 mm LEDs surround the central super-bright-white LED.

84) FIG. 8. Illustrates an electrical schematic 295 for a sub array of light sources in accordance with an embodiment. In this figure, the electrical schematic pertains specifically to an embodiment employing mini-flower mosaic sub array configuration including the nine LEDs detailed in Table 1 above. However, one of skill in the art will recognize that the principle of operation and the parts and components may be adapted to suit any number of other light array configurations within the scope of the claims. In order to permit the emulation of multiple ecological light sources as described above, the electrical network depicted allows the LEDs to be selectively enabled and disabled as well as their intensity to be adjusted by allowing individual variations in the current applied to each LED. Light emitted from the array of light sources is thus configured to be spectrally adjustable and the array of light sources configured to be selectively adjustable in intensity. This may be accomplished using a power supply 296 coupled to adjustable voltage regulators 297 which are in turn coupled to each of respective LEDs 240. Current may be limited to fall within the specifications of the selected LEDs using either a single resistor or multiple parallel-connected resistors that form a current divider network calculated to achieve the necessary current constraints of the selected LEDs.

85) FIG. 9. Illustrates a light panel incorporating a camera system in accordance with an embodiment. A SAOPA further includes an imaging system with a camera 520, which may be affixed to, embedded in, or mounted near light panels 200 and 202, or otherwise positioned such that images or video capture of at least the ocular region of the subject's face may be obtained. Preferably, camera 520 will be capable of full-face capture. Camera(s) will ideally operate at a minimum of 60 frames per second, record rear infrared, and have an image resolution of at least 10 pixels/mm. In the embodiment depicted, three camera lenses are utilized. Camera lens 520 is positioned centrally and configured to capture both eyes and preferably the substantial entirety of the subject's face. Camera lenses 522 and 524 are positioned above camera lens 520 and are positioned in general alignment with the average position of a human subject's eyes so that camera lens 522 may image the left eye of the subject whereas camera lens 524 may image the right eye.

86) In the embodiment depicted, cameras lenses 522 and 524 are implemented using a 50 mm Nativar Lens system (e.g., Thorlabs MVL50M23) to image the eyes, and camera 520 is implemented using a 12 mm Navitar lens system (e.g., Thorlabs MVL12M23 1) to image the face. Both Lens systems may be coupled to the same camera sensor in order to provide the desired field of view. In this embodiment, the UI-3360CP_NIR-GL-Rex.2 from Imaging Development Systems GmbH. The camera utilizes a ⅔″ sensor format, with a sensor size of 11.264 mm×5.948 mm, USB 3.0 interface; 2.23 megapixels, a resolution of 2048×1088 pixels, and supports frame rates of up to 152 frames per second. The camera covers the near infrared spectra and is capable of the desired 60 frames or greater per second for imaging. It is advantageous to block light being shined on subjects' faces by the light source from the camera. In some embodiment, a near-IR bandwidth filter such as a Midwest optics 850 Near-IR Bandpass filter may be incorporated into the camera system. A useful range of this filter is between about 820 nm and 910 nm. The peak transmission of this filter is approximately 90% and it is compatible with 840 nm and 850 nm LEDs.

87) Camera(s) of the imaging system are operatively coupled to a processor and display. The computing system may be integrated into a single device or may be separated (as depicted in FIG. 1 with personal computer 540 being physically separate from camera 520). In either case, sufficient bandwidth should be allowed given the high frame rate of the camera(s). USB protocol or other high bandwidth wired data interfaces such as SATA, SAS, or PCIe or high-speed wireless data communication interfaces such as ANT, UWB, Bluetooth, ZigBee, and Wireless USB may be utilized. In the exemplary embodiment described herein, a 4-port USB 3.1 hub from Point grey with an effective USB bandwidth of Approximately 450 MB/s was utilized as an interface between the camera system and the PC. The PC may be a touch-based computer graphical user interface available from National Instruments, Austin, Tex. and designed to record high resolution infrared video, record light intensity data, track the interval between stimuli, record subject responses, and calculate the light intensity and LED voltage coefficients for generating the stimuli emulating a reference ecological light source (e.g., LED, incandescent, halogen and solar).

88) Computing system 540 (shown in FIG. 1) is programmed to store a series of software instructions that when executed by the processor cause the processor of the SAOPA 100 to effect a testing protocol capable of quantifying a visual photosensitivity threshold of the human subject. As illustrated by the process illustrated in FIG. 10, such a method 700 includes at a step 702 emitting light toward an eye of the human subject at increasing intensities beginning with a least light intensity and gradually increasing toward a greatest light intensity; at a step 704 receiving a stimulus response from the human subject indicating at what intensity the light causes discomfort; and at a step 706 repeating steps 702 and 704 to achieve a plurality of reversals, i.e., a change of the subject's current response is different from the previous stimulus response, changing from yes (positive) to no (negative) or vice versa.

89) To minimize the effects of confounding variables during test administration, in the testing protocol, it is preferred to standardize the procedure by incorporating synthesized speech to administer test instructions and questions throughout all testing stages. The primary guideline is for the subject to indicate after each stimulus whether the light stimulus is uncomfortable by pressing the handheld push-button. Even more preferably, the protocol is automated by software wherein the SAOPA automates the testing procedure. In a preferred embodiment, the automated SAOPA starts with the dimmest light stimulus and is gradually increased; the intensity may be adjusted utilizing the Garcia-Perez staircase technique, which uses unequal ascending and descending steps. Light stimuli are presented for a fixed duration of two seconds with a four second inter-stimulus rest period. During testing, the subject is queried repeatedly if the previous stimulus was uncomfortable. They respond either yes (positive) with a button press or no (negative) with no button press. A subject's discomfort response based on their button press, will either increase or decrease the light intensity for the next stimulus. In a preferred embodiment, the subject's discomfort response is determined using image processing to ascertain a squint response. A response reversal is defined as when a subject's current response is different from the previous stimulus response, changing from yes (positive) to no (negative) or vice versa. The test concludes after response reversals and the visual photosensitivity threshold is calculated from the mean of the 10 response reversals. Additionally, the SAOPA may integrate subject response reliability measure by utilizing catch trials throughout the testing paradigm. Except for the first stimulus, every third stimulus may execute a catch trial. A catch trial is defined as a random repetition of a recently presented stimulus. The subject's response to the previously administered stimulus is compared to that of the catch trial stimulus for consistency, from which a positive/negative inconsistency index score is computed.

90) Software operating on computer system 540 is operable to control light sources 220 to emulate ecological light sources, for example, by operatively controlling the current applied to each of the light sources 220. More specifically, in the embodiment described herein, which implements the 78 mini-flower sub arrays, each subarray is controlled via hardware and software to generate a stimulus emulating four light reference sources, (solar, incandescent, halogen, and LED). Optimal gain coefficients for adjusting the LED's intensity and producing the selected spectra, may derived using a two-phase process. These optimal gain coefficients are incorporated into the control software to generate and control the light emitted by the bi cupola. In a first phase, an initial estimate of LED gain coefficients may be obtained. A Levenberg-Marquardt gradient search algorithm may be used to supply an initial best fit for the light source gain coefficients. The coefficient values may then be sent to an analog voltage output module, such as National Instrument NI-9264 to generate a voltage at the corresponding PCB operational amplifier. The final step in this phase is the signal generated by the light panel was captured by the spectrometer and the resulting spectra is transferred to phase two of this process.

The initial best fit LED gain coefficients estimated in phase one and the resulting spectra are further refined to improve the emulation of the selected reference light source. The difference between the resulting spectra and the selected reference is transferred to the Levenberg-Marquardt gradient search algorithm that generates the optimal coefficients for generating the difference profile. The original gain coefficients are adjusted by these new difference coefficients, and a process similar to phase one begins. These updated coefficient values are sent to the analog voltage output module, which generates a voltage at the corresponding PCB operational amplifier and then the light generated by the light panel is captured by the spectrometer, with resulting spectra compared again to the reference. This closed feedback loop process continues to iterate until the difference between the spectra generated and the selected reference, reach a minimum.

The descriptions herein are not intended to limit the myriad embodiments to one preferred embodiment. To the contrary, it is intended to cover alternatives, modifications, and equivalents as can be included within the spirit and scope of the described embodiments as defined by the appended claims. Further, when a particular feature, structure, or characteristic is described in connection with an embodiment, it is submitted that it is within the knowledge of one skilled in the art to effect such feature, structure, or characteristic in connection with other embodiments whether or not explicitly described.

Furthermore, the Summary and Abstract sections may set forth one or more but not all exemplary embodiments of the present invention as contemplated by the inventors, and thus, are not intended to limit the present invention and the appended claims in any way.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

What is claimed is:

1. An ocular photosensitivity analysis system comprising:
91) a light panel configured to cast light toward an eye of a human subject comprising an array of light sources having different wavelengths selected such that light emitted from the array of light sources combine to emulate a light emission spectra of an ecological light source; and
92) an imaging system comprising a camera configured to capture images of at least a portion of an eye of a human subject in response to exposure to the light emitted from the array of light sources.
93)

[symbol]

2. The ocular photosensitivity analysis system of claim 1 wherein the light emitted from the array of light sources combine to emulate the light emission spectra of an ecological light source selected from at least one of following ecological light sources: solar, LED, incandescent, and halogen.

3. The ocular photosensitivity analysis system of claim 2 wherein the light emitted from the array of light sources is configured to be spectrally adjustable.

4. The ocular photosensitivity analysis system of claim 1 wherein the array of light sources is configured to be selectively adjustable in intensity.

5. The ocular photosensitivity analysis system of claim 1 wherein the array of light sources comprises a plurality of LEDs.

6. The ocular photosensitivity analysis system of claim 1 wherein the light panel is configured in a cupola shape.

7. The ocular photosensitivity analysis system of claim 1 wherein the wavelengths of the light sources are selected from a group comprising about 370 nm, about 395 nm, about 420 nm, about 470 nm, about 505 nm, about 545 nm, about 630 nm, about 660 nm, and about 735 nm.

8. The ocular photosensitivity analysis system of claim 1 wherein the wavelengths of the light sources are selected from a group comprising about 395 nm, about 440 nm, about 480 nm, about 520 nm, about 555 nm, about 590 nm, about 650 nm, about 670 nm, and about 720 nm.

9. The ocular photosensitivity analysis system of claim 5 wherein at least a plurality of the LEDs have a size of about 5 mm.

10. The ocular photosensitivity analysis system of claim 1 wherein the light sources are embedded into the light panel in a plurality of sub arrays.

11. The ocular photosensitivity analysis system of claim 10 wherein each sub array may be chosen to exhibit a hexagonal configuration to optimize fill factor in the array.

12. The ocular photosensitivity analysis system of claim 1 wherein the spectral characteristics of each of the light sources may be selected to permit metameric representation across a wide color gamut.

13. The ocular photosensitivity analysis system of claim 10 wherein the light sources in each of the sub arrays are arranged in a mosaic pattern wherein each of the light sources in each of the sub arrays emits a light of a different wavelength.

14. The ocular photosensitivity analysis system of claim 13 wherein the mosaic pattern comprises a central light source surrounded by a plurality of peripheral light sources.

15. The ocular photosensitivity analysis system of claim 10 wherein a least one of the sub arrays comprises at a super bright white LED.

16. The ocular photosensitivity analysis system of claim 13 wherein the light sources in each of the subarrays are positioned in a subarray cupola that focuses the LEDs at a specified distance.

17. The ocular photosensitivity analysis system of claim 16 wherein the specified distance is between about 350 mm and 500 mm.

18. The ocular photosensitivity analysis system of claim 1 further comprising a second light panel substantially mirroring the configuration of the light panel.

19. The ocular photosensitivity analysis system of claim 18 wherein light panel and the second light panel are each configured in a cupola shape to form a bicupola arrangement.

20. The ocular photosensitivity analysis system of claim 18 wherein the light panel and the second light panel each have radii that points to an average interpupillary distance of about 32 mm from the center of the face of the human subject.

21. The ocular photosensitivity analysis system of claim 1 wherein the camera is positioned at approximately the center of the light panel and approximately at the level of the eye of the human subject.

22. The ocular photosensitivity analysis system of claim 1 wherein the camera is a video camera capable of capturing at least about 60 frames per second.

23. The ocular photosensitivity analysis system of claim 1 further comprising a second and a third camera wherein: the camera is configured to capture images including a section of the face of the human subject comprising at least a portion of both eyes of the human subject; the second camera is configured to capture images including a left eye of the human subject; and the third camera is configured to capture images includes a right eye of the human subject.

24. The ocular photosensitivity analysis system of claim 1 wherein the imaging system further comprises a near-IR bandpass filter having a filter range of between about 820 nm to 910 nm.

25. The ocular photosensitivity analysis system of claim 1 further comprising a processor and a memory wherein the memory is programmed to store a series of software instructions that when executed by the processor cause the ocular photosensitivity analysis system to effect a testing protocol capable of quantifying a visual photosensitivity threshold of the human subject.

26. A method of quantifying a visual photosensitivity threshold of a human subject employing an ocular photosensitivity analysis system as in any of claims 1-25, the method comprising:
94) emitting light toward an eye of the human subject at increasing intensities beginning with a least light intensity and gradually increasing toward a greatest light intensity;
95) receiving a stimulus response from the human subject indicating at what intensity the light causes discomfort;
96) repeating steps 1 and 2 to achieve a plurality of reversals, i.e., a change of the subject's current response is different from the previous stimulus response, changing from yes (positive) to no (negative) or vice versa.

27. A method of quantifying a visual photosensitivity threshold of a human subject employing an ocular photosensitivity analysis system as in any of claims 1-25, the method comprising:
1) emitting light toward an eye of the human subject at increasing intensities beginning with a least light intensity and gradually increasing toward a greatest light intensity;
2) inferring discomfort from a quantitative measure of squint response;
3) repeating steps 1 and 2 to achieve a plurality of reversals, i.e., a change of the subject's current response is different from the previous stimulus response, changing from yes (positive) to no (negative) or vice versa.
